(19) United States
(12) Patent Application Publication
(10) Pub. No.: US 2023/0338029 A1
(43) Pub. Date: Oct. 26, 2023

(54) NERVE GROWTH SYSTEM

(71) Applicant: Pioneer Neurotech Inc., Sudbury, MA (US)

(72) Inventors: Mario I. Romero-Ortega, Coppell, TX (US); David Constantine, Sudbury, MA (US); Jeffrey Petruska, Sudbury, MA (US)

(21) Appl. No.: 18/131,345

(22) Filed: Apr. 5, 2023

Related U.S. Application Data
(60) Provisional application No. 63/327,464, filed on Apr. 5, 2022.

(51) Int. Cl. A61B 17/11; A61L 27/38; A61L 27/54; A61L 27/58; A61L 27/24

(52) U.S. Cl. CPC A61B 17/1128; A61L 27/383; A61L 27/54; A61L 27/58; A61L 27/24; A61L 2430/32; A61L 2300/414

(57) ABSTRACT

A nerve regeneration system includes a nerve guide having a proximal end and a distal end. The system includes nerve growth factor configured to enhance the growth of axons and associated nerve tissue. The nerve growth factor has a first concentration nearer to a proximal end and a second growth factor concentration nearer to a distal end. The second growth factor concentration is higher than the first growth factor concentration. The system includes myelination factor configured to enhance myelination of the grown axons. The myelination factor has a first myelination factor concentration nearer to the proximal end, a third myelination factor concentration nearer to the distal end, and a second myelination factor concentration between the first myelination factor concentration and the third myelination factor concentration. The second myelination factor concentration is higher than the first myelination factor and higher than the third myelination factor concentration.

### PRIORITY

This patent application claims priority from provisional U.S. patent application No. 63/327,464, filed Apr. 5, 2022, entitled, “APPARATUS FOR NERVE GROWTH,” and naming Mario Romero-Ortega, David Constantine, and Jeffrey Petruska as inventors, the disclosure of which is incorporated herein, in its entirety, by reference.

### FIELD OF THE INVENTION

Illustrative embodiments of the invention generally relate to nerve growth and, more particularly, various embodiments of the invention relate to enhancing nerve regrowth using various gradients.

### BACKGROUND OF THE INVENTION

Nerves are the major structures of the Peripheral Nervous System (“PNS”), which generally includes relevant structure outside the brain and spinal cord (i.e., outside of the Central Nervous System, or “CNS”). Nerves are primarily made up of the wirelike extensions of sensory, autonomic, and motor neurons—the axons. As such, a nerve is a collection of axons, usually one per neuron. Unlike blood vessels, which also extend throughout the entire body but are made up of many individual cells that link together to form tubes to carry blood, the full length of an axon is part of a single neuron. Axons are wrapped intermittently along their course by supporting cells known as “Schwann cells.” Some of these produce an insulation called myelin, the physical structure vital to enable effective conduction of the electrical signals carried by the axons (action potentials; APs). Axons also carry chemical signals in both directions between the neuron cell body (soma) and the target tissue (e.g., spinal cord, skin, muscle, nerve, blood vessels, viscera). In addition, nerves are endowed with vascular supply to provide nutrients and immune surveillance and to remove waste.

Peripheral nerve (“PN”) injuries occur in hundreds of thousands of people each year as a result of trauma, disease, or other procedures, such as radiotherapy and radical prostatectomy. Many of these cases have damage severe enough to create a gap between the ends of the severed nerve that cannot be spanned simply by acutely stretching the ends to join them directly. Some cases have gaps so large that there is currently no treatment that can offer reasonable hope of any recovery, so the nerve is left unrepaired, often leading to formation of a painful neuroma.

### SUMMARY OF VARIOUS EMBODIMENTS

In accordance with an embodiment of the invention, a nerve regeneration system includes a nerve guide having a proximal end and a distal end. The nerve guide has a housing with one or more ducts. The one or more ducts extend in a direction that is substantially transverse to a longitudinal axis of the nerve guide. A matrix material is disposed within the housing. The matrix material defines one or more channels extending in a direction that is substantially along the longitudinal axis of the nerve guide. The system includes nerve growth factor configured to enhance the growth of axons and associated nerve tissue. The nerve growth factor has a first concentration nearer to the proximal end and a second growth factor concentration nearer to the distal end. The second growth factor concentration is higher than the first growth factor concentration. The system includes myelination factor configured to enhance myelination of the grown axons. The myelination factor has a first myelination factor concentration nearer to the proximal end, a third myelination factor concentration nearer to the distal end, and a second myelination factor concentration between the first myelination factor concentration and the third myelination factor concentration. The second myelination factor concentration is higher than the first myelination factor and higher than the third myelination factor concentration.

In various embodiments, the channels include a channel material containing the nerve growth factor and/or the myelination factor. In some other embodiments, the matrix may include the matrix having the nerve growth factor and/or the myelination factor. The channel is configured so that axons can grow therethrough. The matrix is configured so that axons do not grow therethrough initially.

Among other things, the housing may include an indicator that indicates a proper orientation of the device, as well as a nerve coupling portion.

In some embodiments, the first nerve growth factor concentration is at the proximal end of the channel and the second nerve growth factor concentration is near the distal end of the channel. The first myelination factor concentration may be near the proximal end of the channel, and the second myelination factor concentration may be near the middle of the channel, and the third myelination factor is near the distal end of the channel.

In various embodiments, the channel has a substantially linear gradient of concentration of growth factor along the longitudinal axis of the channel from the first growth factor concentration to the second growth factor concentration. In some embodiments, the channel has a substantially central peak gradient of concentration of growth factor along the longitudinal axis of the channel from the first growth factor concentration to the second growth factor concentration.

The device may be configured to release the nerve growth factor from a biodegradable polymer at a first time for a first duration. The device may also be configured to activate the myelination factor at a second time for a second duration. In various embodiments, the second time is after the first time, and the first duration temporally overlaps with the second duration. The second time may be at least 1 week after the first time. In some embodiments, the second time may be at least 2 weeks after the first time. Furthermore, the second time may be about 20 days after the first time.

A release rate of the nerve growth factor may increase from the first growth factor concentration to the second growth factor concentration. The channels may include a secondary nerve growth factor. The channel has a first secondary growth factor concentration nearer to the proximal end and a second secondary growth factor concentration nearer to the distal end. The second secondary growth factor concentration may be higher than the first secondary growth factor concentration.

Among other things, the growth factor and/or the secondary growth factor may include nerve growth factor (NGF), brain-derived neurotrophic factor (BDNF), glial cell line-derived neurotrophic factor (GDNF), insulin-like growth factor-1 (IGF-1), and ciliary neurotrophic factor (CNTF), pleiotrophin (PTN) (also known as heparin-binding growth factor 8, HBGF-8). The myelination factor and/or the secondary myelination factor may include Neuregulin-1 (NRG1 also known as acetylcholine receptor-inducing activity (ARIA), breast cancer cell differentiation factor p45, glial growth factor, heregulin (HRG), Neu differentiation factor, sensory and motor neuron-derived factor), platelet-derived growth factor (PDGF), fibroblast growth factor (FGF), sonic hedgehog (Shh).

In accordance with another embodiment, a method regenerates a damaged nerve having a nerve gap of at least 4 cm. The method provides a damaged nerve having a proximal trunk and a distal trunk. A first growth factor concentration is provided nearer to the proximal trunk and a second growth factor concentration is provided nearer to the distal trunk. The second growth factor concentration is higher than the first growth factor concentration. A first myelination factor concentration is provided nearer to the proximal trunk. A third myelination factor concentration is provided nearer to the distal trunk. A second myelination factor concentration is provided between the first myelination factor concentration and the third myelination factor concentration. The second myelination factor concentration is higher than the first myelination factor concentration and higher than the third myelination factor concentration. The method restores function of the nerve over a gap of at least 4 cm.

Among other ways, the providing the growth factor begins at a first time. Similarly, providing the myelination factor begins at a second time after the axons have regenerated to the point at which myelination is possible. In various embodiments, the first time is at least 1 week before the second time. Furthermore, the nerve growth factor is provided over a first duration, and the myelination factor is provided over a second duration. The second duration may be longer than the first duration.

In various embodiments, at least 60% of a total concentration of myelination factor is released within 200 hours of coupling the device to the nerve. Furthermore, the concentrations provided may be between 1 ng/mL-100 ng/mL.

Some embodiments may provide a nerve guide having a proximal end and a distal end. The nerve guide may include a biodegradable housing with one or more ducts. The one or more ducts may extend in a direction that is substantially transverse to a longitudinal axis of the nerve guide. The nerve guide may also include a matrix material disposed within the housing. The matrix material may define one or more channels extending in a direction that is substantially along the longitudinal axis of the nerve guide.

A first growth factor concentration may be nearer to the proximal trunk and a second growth factor concentration may be nearer to the distal trunk. The second growth factor concentration may be higher than the first growth factor concentration. The method may provide the myelination factor at a third time that is at least 1 week before the second time.

Various embodiments may provide a secondary nerve growth factor. A first secondary growth factor concentration may be nearer to the proximal end and a second secondary growth factor concentration may be nearer to the distal end. The second secondary growth factor concentration may be higher than the first secondary growth factor concentration. Similarly, a secondary myelination factor may be provided.

In various embodiments, the matrix is formed from agarose. The channel may be include collagen.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a cross-sectional view of a nerve in accordance with illustrative embodiments.

FIG. 2 schematically shows a process of peripheral nerve injury in accordance with illustrative embodiments.

FIG. 3A schematically shows a partially transparent perspective view of a nerve regeneration device in accordance with illustrative embodiments. FIG.

3B schematically shows a cross-sectional view of the device of FIG. 3A.

FIGS. 4A-4C schematically show details of the channels 52 in accordance with illustrative embodiments.

FIG. 5A graphically shows an example of the concentration of the nerve growth factor from the proximal end to the distal end in accordance with illustrative embodiments.

FIG. 5B graphically shows examples of a release profile of the nerve growth factor from the proximal end to the distal end in accordance with illustrative embodiments.

FIG. 5C graphically shows examples of a release duration of the nerve growth factor from the proximal end to the distal end in accordance with illustrative embodiments.

FIG. 6A graphically shows an example of the concentration of the myelination factor from the proximal end to the distal end in accordance with illustrative embodiments.

FIG. 6B graphically show examples of a release profile of the myelination factor from the proximal end to the distal end in accordance with illustrative embodiments.

FIG. 6C graphically shows examples of a release duration of the myelination factor from the proximal end to the distal end in accordance with illustrative embodiments.

FIG. 6D schematically shows an example of a cumulative release pattern for the myelination factor over time in accordance with illustrative embodiments.

FIG. 6E schematically shows an example of a cumulative release pattern for the nerve growth factor and the myelination factor in accordance with illustrative embodiments.

FIG. 7 shows a process of regenerating the nerve in accordance with illustrative embodiments.

FIG. 8 schematically shows a channel with an exemplary concentration, release time, and release rate for each the nerve growth factor and the myelination factor, respectively, in accordance with illustrative embodiments.

### DESCRIPTION OF ILLUSTRATIVE EMBODIMENTS

In illustrative embodiments, a damaged (e.g., cut) nerve is regenerated, particularly along long gaps (e.g., 2 cm, 3 cm, 4 cm, and greater). The nerve is regenerated by providing a nerve growth factor and a myelination factor (collectively, the factors) within the gap. The factors are each provided in a given concentration gradient with the gap, respectively. The factors are released at different times relative to one another. Additionally, in some embodiments, each of the factors has a different time release pattern/gradient along the length of the gap.

Furthermore, various embodiments include a device that couples with the proximal trunk and the distal trunk of the nerve. The device is configured to contain the nerve growth factor and the myelination factor in predetermined concentrations. The factors are configured to provide the predetermined release patterns when implanted (e.g., gradients). In particular, the device is configured to provide a given release pattern for each of the factors throughout the growth cycle of the nerve so as to advantageously enhance growth and myelination of the damaged nerve, such that axons of the nerve may be grown over long gaps. Effective use of this combination device enables growth across shorter and longer nerve gaps. Details of illustrative embodiments are discussed below.

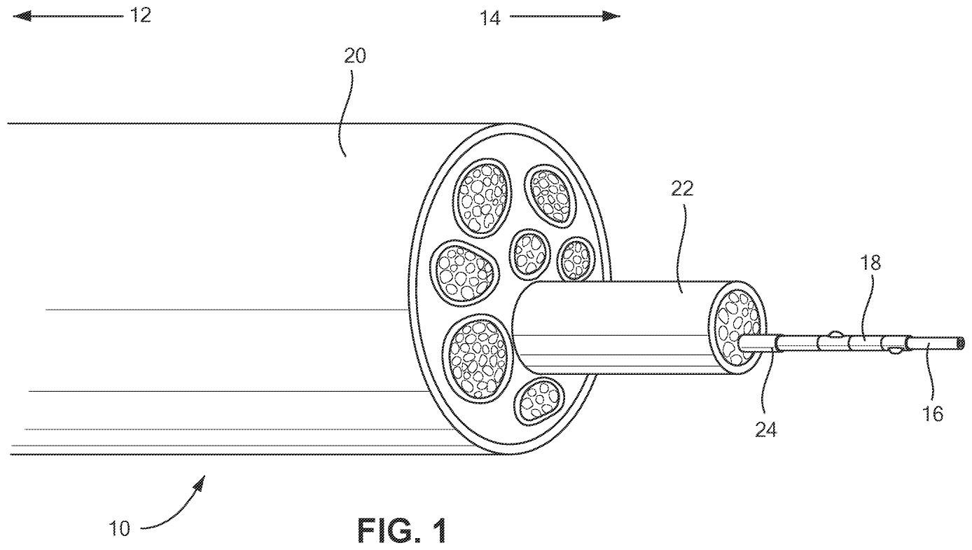

FIG. 1 schematically shows a cross-sectional view of a nerve 10 in accordance with illustrative embodiments. Discussion of various embodiments relates to peripheral nerves 10. However, illustrative embodiments may also include central nerves 10.

The nerve 10 is an enclosed, cable-like bundle of nerve fibers (referred to as axons 16). Each axon 16 is an extension of an individual neuron, along with other supportive cells such as some Schwann cells that coat the axons 16 in myelin 18. As used herein, the proximal end 12 refers to the end of neuron that is closer to the spine (e.g., the nucleus of the neuron). Accordingly, the axon 16 grows outwardly and away from the neuron towards the distal end 14 (e.g., closer to the innervated muscle).

Each nerve 10 is covered on the outside by a dense sheath of connective tissue referred to as the epineurium 20. Beneath the epineurium 20 is a layer of fat cells, the perineurium 22, which forms a generally complete sleeve around a bundle of axons 16 (referred to as fascicles). The endoneurium 24 (also called endoneurial channel, endoneurial sheath, endoneurial tube, or Henle's sheath) is a layer of delicate connective tissue around the myelin 18 sheath of each myelinated axon 16 in the peripheral nervous system. In undamaged nerves 10, the endoneurium 24 forms an unbroken tube from the surface of the spinal cord to the level where the axon 16 synapses with its muscle fibers, and/or ends in sensory receptors. The endoneurium 24 consists of an inner sleeve of material called the glycocalyx and an outer, delicate, meshwork of collagen fibers. Although not shown in FIG. 1, the nerve 10 also includes blood vessels throughout.

As referenced previously, the axons 16 are wrapped intermittently along their course by supporting cells known as Schwann cells. Some of these cells produce an insulation called myelin 18, the physical structure of which is vital to enable effective conduction of the electrical signals carried by the axons (action potentials; APs). Regenerating axons 16 may encounter regions of implanted conduit or native denervated nerve which have no, or few, Schwann cells. Without Schwann cells, the portions of the axons 16 in these areas are not metabolically-supported or myelinated by Schwann cells. As discussed herein, various signals can enhance the migration of Schwann cells into implanted conduits, encourage and enhance their proliferation in conduits and nerve, and prevent or reverse their senescence. Various signals can also influence the properties of myelination—its quality (compaction, thickness) and spacing—which greatly influence the fidelity, speed, and frequency of APs which can be conducted by the axon 16, and therefore its efficacy in driving muscle contraction (for motor axons) and sensation (for sensory axons).

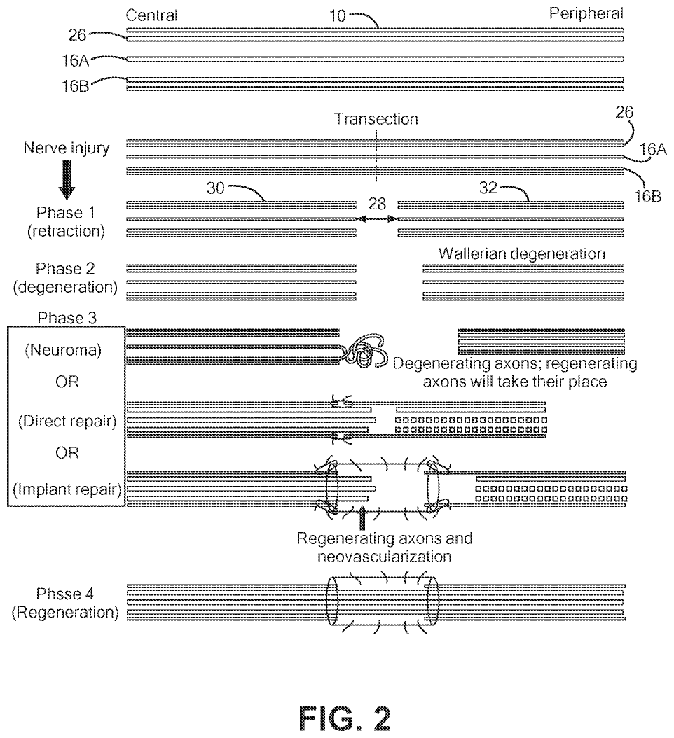

FIG. 2 schematically shows a process of peripheral nerve 10 injury in accordance with illustrative embodiments. The nerve may include vasculature 26, sensory axons 16A, motor axons 16B, and/or autonomic axons. Peripheral nerve 10 injuries occur in hundreds of thousands of people each year as a result of trauma, disease or other procedures including radiotherapy and radical prostatectomy. Many of these cases have damage severe enough to create the aforementioned gap 28 between the ends of the severed nerve 10 that cannot be spanned simply by acutely stretching the ends to join them directly. These cases generally require insertion of some assistive device to bridge the gap 28 in an effort to restore function. Some gaps 28 are so large that the state of the art offers no reliable treatment for recovery, so the nerve 10 is left unrepaired, often leading to formation of a painful neuroma.

As shown in Phase 1, in nerve injury conditions in which axons 16 are severed, the nerve 10 can has a proximal trunk 30 and a distal trunk 32. The portion of the axon 16 that is separated from the nucleus (e.g., the portion of the axons 16 that are associated with the distal trunk 32) dies, degrades, and the debris is removed by immune cells. Various embodiments support/enhance the growth of axons 16 from the proximal trunk. Because axons 16 are extensions from the nucleus, for them to become re-connected to their end target (e.g., muscle), the axons 16 grow from the point where they were severed back to their target (but do not reconnect with the distally disconnected portion of the axon 16, which is removed by immune cells). This re-connection, shown at phase 4, is the first step in restoring the electrical and chemical communication of the nerve. In humans, the distance the nerve needs to grow, can be over 1 meter long. The distance the axons 16 ultimately grow in a successful regeneration includes both the gap 28 distance and the distance to the final terminal target (e.g., muscle, skin, viscera). The overall distance can be over 1 meter, but the gap 28 caused by axon 16 injury is usually considerably shorter.

Nerve regeneration can require a length of time that makes distal nerve tissue and target tissues unreceptive to regenerating axons, preventing successful reconnection and recovery of function. Illustrative embodiments provide chemical factors that provide signals to enhance the speed of axon 16 regeneration and myelination to enable a greater distance to be covered before the denervated nerve and target tissues become unreceptive.

The effective and/or correct connection between specific neuron type (sensory, autonomic, or motor) and the specific body location (skin area, visceral structure, muscle, etc.) is established during development, and each axon 16 is endowed with the connective tissue encasement (endoneurium). If a nerve injury severs an axon 16 but not the endoneurium 24, a regenerating axon 16 can generally reach its original target. However, when there is a nerve injury that opens the endoneurium 24, regenerating axons 16 may not be able to find their original target. The inventors recognized that certain signals can enhance the axonal target-finding such that an increased number of axons 16 can more accurately connect to their proper target tissue type.

Nerve 10 injury recruits immune cells to the site of injury. Some kinds of immune cells can induce failure of axonal regeneration, while others can enhance it. Illustrative embodiments provide factors that produce signals to modulate or direct the population of immune cells so that they migrate into the injury/implant/regeneration area, or affect a change in those that are present in the injury/implant/regeneration area. Consequently, such a process and/or device is configured for modulating axonal regeneration.

Nerve 10 injury also sever axons 16 and blood vessels in the nerve. Although axons 16 may have the capability of regenerating into implanted conduits or native denervated nerve segments, without the concomitant assembly of new lengths of blood vessels, axon 16 growth stalls. Illustrative embodiments therefore produce and/or enhance various signals and mechanical structures to encourage neovascularization and enable infiltration of vessels into implanted nerve guide device 34 and regenerated nerve tissue.

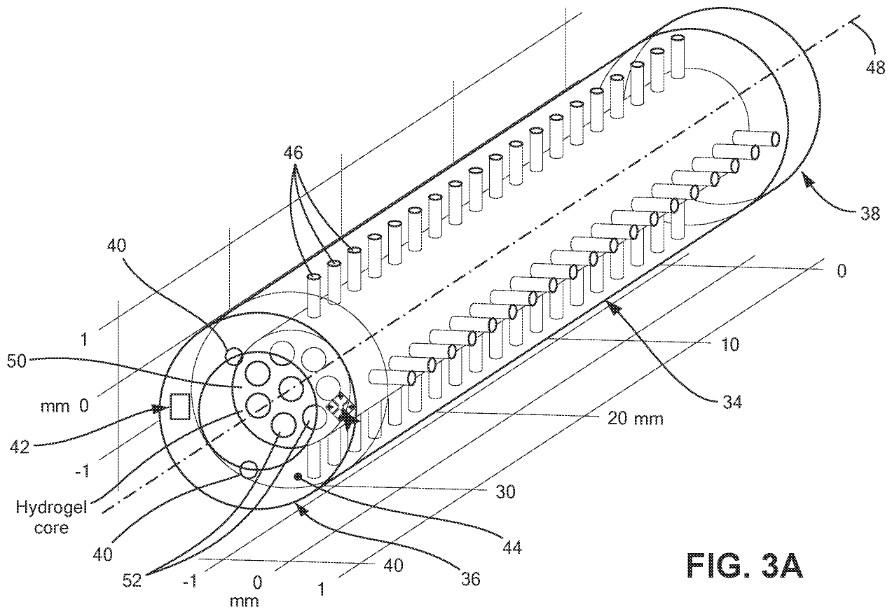

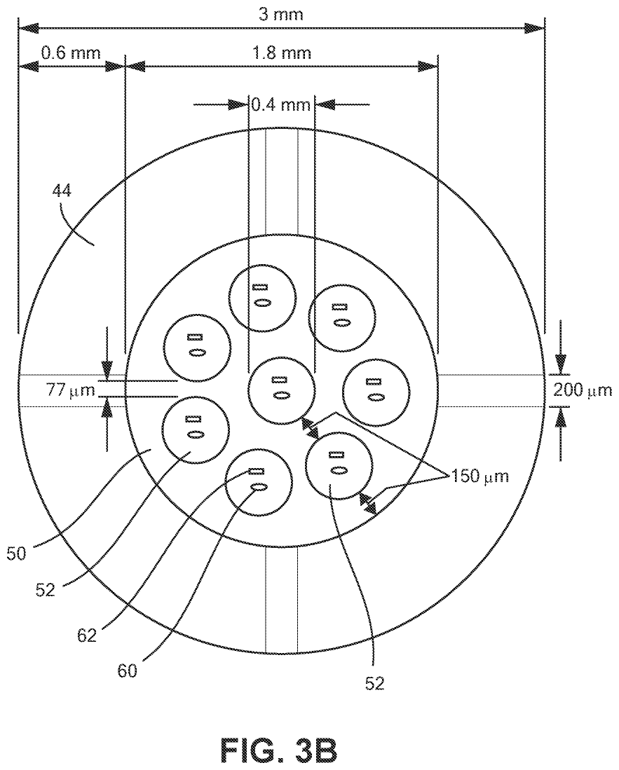

FIG. 3A schematically shows a partially transparent perspective view of the nerve regeneration device 34 in accordance with illustrative embodiments. FIG. 3B schematically shows a cross-sectional view of the device 34 of FIG. 3A. The device 34 functions as a guide and scaffolding for the axons 16 and associated nerve tissue as it grows towards the distal end 14. The device 34 is configured to assist nerves 10 of various sizes to regenerate and remyelinate axons 16 across small to large gap 28 sizes. It should be noted FIGS. 3A-3B show a variety of dimensions for the device 34. These dimensions are not intended to limit various embodiments, and merely show dimensions for one example of the device 34.

The size of the device 34 is selected to be suitable for the given gap 28 length and nerve 10. For the example of nerve 10 repair, the diameter of the device 34 is suitable to interface with, and attach to, the portion of the nerve 10 to be repaired (e.g., typically the ends of the nerves 10). For other tissues, the device 34 face may suitably match the face of the tissue. For instances in which the device 34 is intended to span separated aspects of the tissue—for example the cut ends of a nerve—the device may be sufficiently-sized to span the separated aspects of the tissue. In various embodiments, a variety of sizes of device 34 may be provided suitable to a variety of sizes of nerves 10.

The device 34 contains the nerve growth factor 60 (e.g., GDNF) and the myelination factor 62 (e.g., NRG1) in concentrations that are configured to have specific release patterns that assist in regeneration and/or myelination of the axons 16 of the nerve 10. Thus, appropriate orientation of the device 34 when coupled with the nerve 10 is significant to optimal outcomes in various embodiments. The device 34 has a proximal end 36 and a distal end 38. The proximal end 36 of the device 34 is configured to couple with the proximal trunk of the nerve 10. Similarly, the distal end 38 of the device 34 is configured to couple with the distal trunk 32 of the nerve 10. To that end, the device 34 may include nerve coupling portions 40 (e.g., clamps and/or eyelets for receiving sutures) to assist with coupling to the nerve 10. Furthermore, the device 34 may include an indicator 42 that indicates to the medical professional the proper intended orientation of the device 34 relative to the nerve 10 (e.g., a marking that indicates which end of the device 34 couples to the proximal trunk 30 and/or the distal trunk 32).

The device 34 has an outer housing 44 preferably formed from a biodegradable material. In some embodiments, the housing 44 may have the shape of a cylindrical tube. However, other embodiments may shape the housing 44 in various ways. For example, the housing 44 may have a substantially cuboid or hexagonal prism shape, among other things. Furthermore, the outer surfaces preferably are smooth without sharp edges. In various embodiments, the housing 44 defines a central or longitudinal axis 48 extending from the proximal end to the distal end of the housing 44.

The housing 44 has a plurality of ducts 46 extending therethrough (e.g., in a direction that is substantially transverse to the longitudinal axis 48). The ducts 46 fluidly couple an exterior of the device 34 within an interior matrix 50 and/or channels 52. Accordingly, the ducts 46 provides for infiltration of neovascular structures to provide blood supply to the cells/tissues inside the channels 52/matrix 50. The ducts 46 may also assist in reconstruction of the perineurium and the epineurium.

Within the housing 44 is the matrix 50 that defines (e.g., by the absence of matrix material) a plurality of channels 52 for axon 16 growth. The matrix 50 is formed of a porous material with pores that allow gas and nutrients to pass, but are generally not large enough for axon 16 growth (although they may become large enough after sufficient degradation of the matrix 50 over time). Accordingly, the device 34 is configured so that axons 16 grow within the channel 52 and not within the matrix 50. In various embodiments, the matrix 50 may be formed from, for example, 1.5%-2.5% agarose gel.

The housing 44 provides a rigid structure relative to the matrix 50 that supports the structure of the matrix 50. The housing 44 material(s) can be selected to provide additional features, such as mechanical strength and attachment-points for things such as surgical suture and/or for biological integration. The housing 44 may contain internal slots so that there are points/channels of separation from the matrix 50 that continue from the ducts 46 to enable cells/fluids/etc. from the outside of the device 34 access to much of the inside of the housing 44 and outside of the matrix 50.

The matrix 50 defines one or more of the channels 52. FIGS. 3A-3B show the device 34 having 8 channels. However, various embodiments may have one or more channels 52. Furthermore, the channels 52 may have a variety of cross-sectional shapes (e.g., circular, rectangular, etc.) and dimensions. Inside of the channel 52 is a biological gel formed materials that provide a permissive growth environment for many types of cells (e.g., collagen type 4). Preferably, the channel 52 is formed from material having viscoelastic properties and nutrients that are suitable for tissue repair.

In illustrative embodiments, the minimum inner dimension of the channels in this implementation is 40-50 microns, and the maximum inner dimension of the channels 52 may be 1.0 to 2.0 millimeters. In addition, or alternatively, the matrix 50 may have between one and multiple channels (e.g., 8, 12, 15, 20). The total number of channels 52 is a function of the size of the overall device 34 and its goals. The spacing between channels 52 (i.e., the amount of matrix 50 between channels), is a function of the matrix 50 material and properties. There is enough matrix 50 between channels to provide a durable, flexible (as needed) channel 52 structure for the intended functional endurance of the device 34. FIG. 1D schematically shows the matrix 50 material inside the housing 44, with channels 52 within the matrix.

In various embodiments, the matrix 50 and/or the channels 52 contain the factors 60, 62 therein. The factors may be provided in the form of Polylactic-co-glycolic acid) (PLGA) microparticles, among other things. In some embodiments, the factors 60, 62 may be distributed throughout the channel 52 in the desired spatial concentrations. Additionally, or alternatively, the factors 60, 62, may be embedded in the matrix 50 and may diffuse into the channel 52 over time. Furthermore, some embodiments may include a fiber coiled around the exterior of at least one channel 52, wherein the fiber comprises an active factor that is operable to diffuse into the interior of the channel 52. The concentration of the factors 60, 62 may be measured over some spatial resolution (e.g., unit volume), such as 500 cubic microns. In various embodiments, the concentration of the factors 60, 62 may be continually increasing along the length of the device, such that a gradient is formed. The device may include the nerve growth factor 60 in an increasing gradient from a first concentration 60A to a second concentration 60B. The device may include the myelination factor 62 in an increasing gradient from a first concentration 62A to a second concentration 62B, and then a decreasing gradient from the second concentration 62B to a third concentration 62C. In various embodiments, the concentrations of the factors 60 may then be released in the channel 52 to provide a similar concentration gradient (even if temporarily because of diffusion in the channel 52).

Although the matrix 50 is described as being within the housing 44, it should be understood that some embodiments may not include the outer housing 44. Instead, the channels 52 may be formed and held together by the matrix 50 (at least temporarily). The matrix 50 may include the ducts 46 and/or have pores sufficiently large for passage of nutrients and neovascular structures.

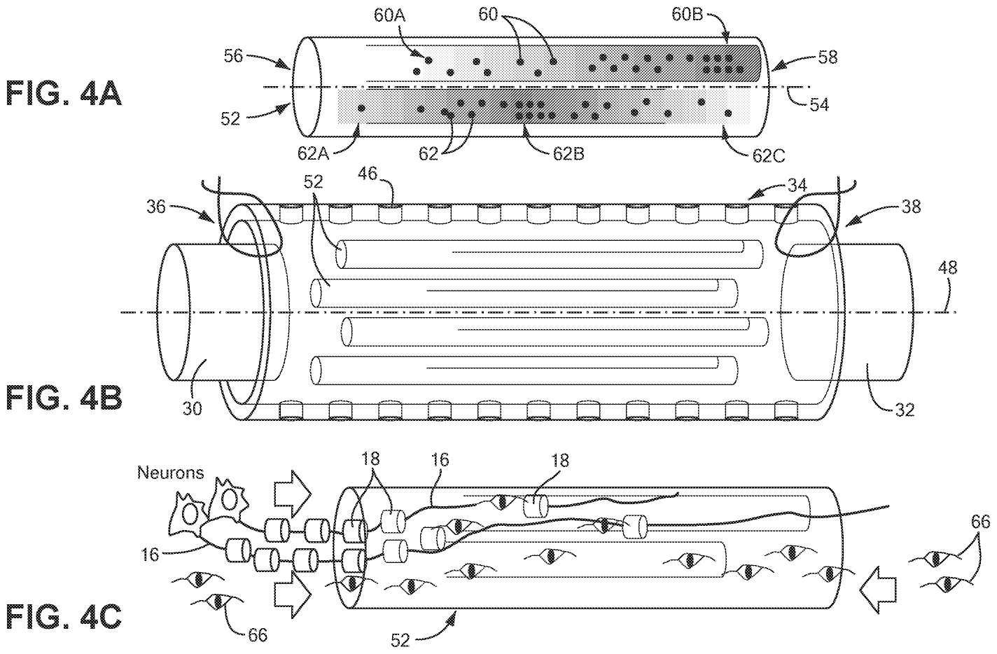

FIGS. 4A-4C schematically show details of the channels 52 in accordance with illustrative embodiments. In particular, FIG. 4A shows an enlarged view of a single channel 52 of the device 34 of FIG. 4B. Although the growth factor 60 and the myelination factor 62 are shown as being on different sides of the channel 52 this is merely for clarity purposes and not intended to limit various embodiments. Accordingly, in practice, the myelination factor 62 and growth factor 60 may be intermingled within the channel 52. However, in some embodiments, the myelination factor 62 may be inside of a separate channel 52 from the nerve growth factor 60.

In various embodiments, the device 34 is configured to release at least two different types of factors 60, 62 within the channel 52. As shown, the two different types of factors 60, 62 are positioned within the channel 52. However, as discussed previously, other embodiments may include the factors 60, 62 in the matrix 50, the housing 44, and/or in fibers surrounding the channel 52. In various embodiments, the factors 60, 62 may be suspended in the channel 52 by freezing. In some other embodiments, the factors 60, 62 may be incorporated in the matrix 50 and released as the matrix 50 dissolves.

In various embodiments, the two factors include: a nerve growth factor 60 configured to enhance the growth of axons 16 and associated nerve tissue, and a myelination factor 62 configured to enhance myelination of the grown axons 16. Furthermore, the myelination factor may aid in axon sorting. The factors 60, 62 may be configured to have various release profiles that aid in the growth and myelination of axons 16.

FIG. 4A shows an exemplary concentration profile for the factors 60, 62 in the channel 52 in accordance with illustrative embodiments. The concentration profile changes relative to a longitudinal axis 54 of the channel 52, which is substantially parallel with the longitudinal axis 48 of the device 34. The channel 52 has a first nerve growth factor concentration 60A near a proximal end 56 and a second nerve growth factor concentration 60B near a distal end 58. In various embodiments, the concentration of the nerve growth factor 60 increases from the first concentration 60A to the second concentration 60B along the longitudinal axis 54 from the proximal end 56 to the distal end 58. Various embodiments therefore provide an increasing concentration gradient from the proximal end 56 to the distal end 58.

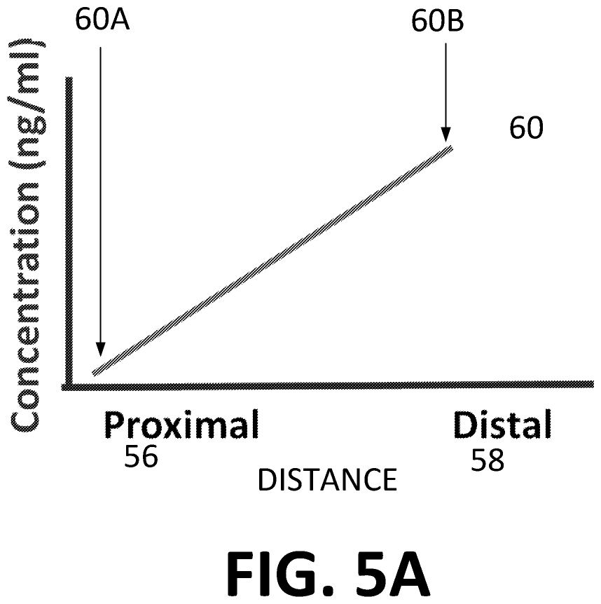

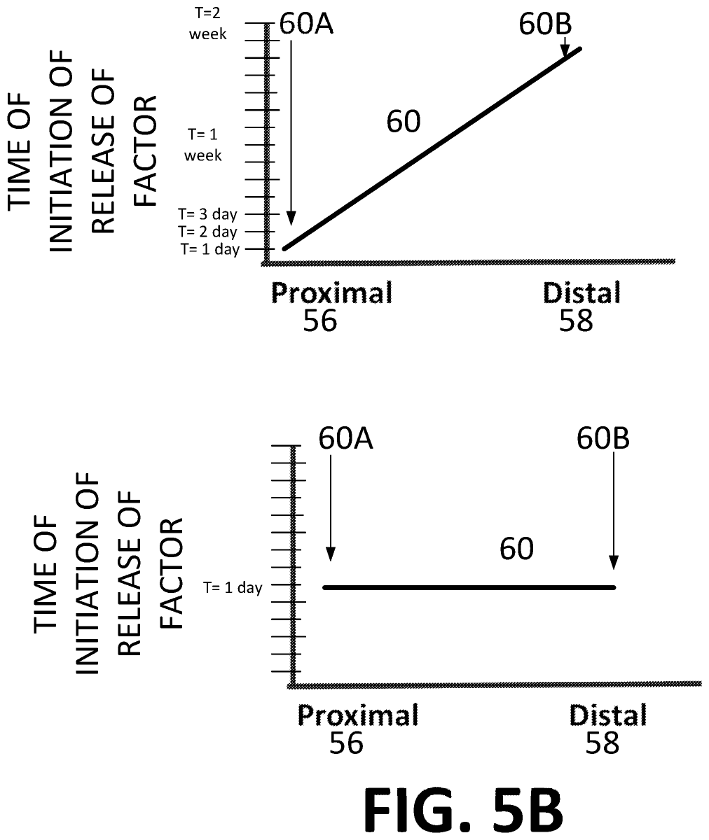

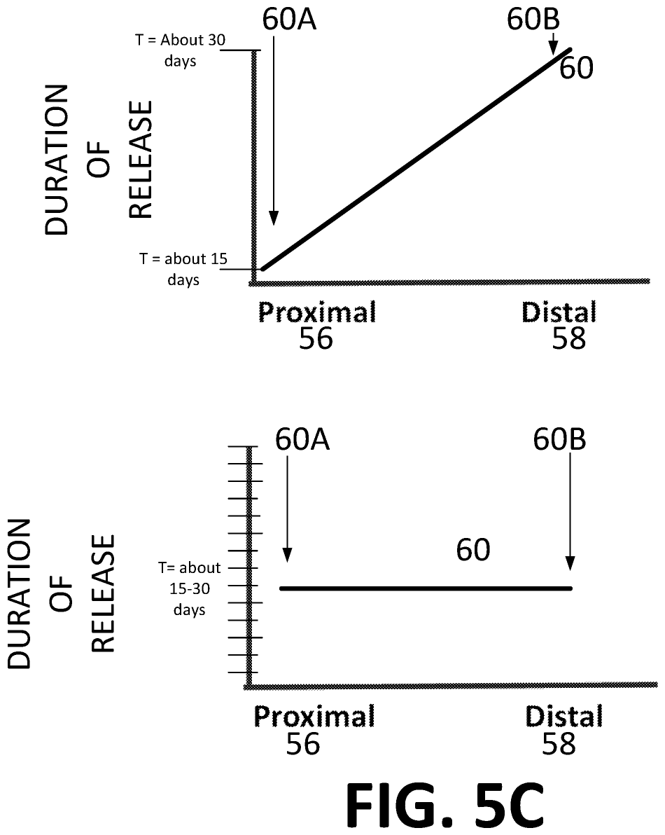

FIG. 5A graphically shows an example of the concentration of the nerve growth factor 60 from the proximal end towards the distal end in accordance with illustrative embodiments. For the nerve growth factor 60, the X-axis distance depends on the length of the channel 52. However, the first concentration 60A may be between about 0 mm and about 0.5 mm from the proximal end 56. As an example, the second concentration 60B may be about 30 mm (e.g., at or near the distal end 58). However, it should be understood that the distance may be longer or shorter in various embodiments. The first concentration 60A may have a concentration of growth factor 60 between about 0 and about 5 ng/ml. The second concentration 60B may have a concentration of growth factor 60 of between about 90 and about 100 ng/ml.

While addition of the nerve growth factor 60 independently assists with growth of axons 16, the inventors discovered that the longitudinally increasing concentration gradient for nerve growth factor 60 improves axon 16 regeneration as compared to a substantially constant distribution of nerve growth factor 60 along the longitudinal axis 54. Without wishing to be limited by any particular theory, the inventors believe, but have not confirmed, that the increased axon 16 growth rate is because axons 16 tend to grow in a direction from areas of low concentration to high concentration of nerve growth factor 60.

Furthermore, axon 16 growth is a slow process. Simultaneous release of nerve growth factor 60 throughout the entire length of the channel 52 may undesirably leads to premature degradation of the growth factor 60 before achieving intended effects on the growing axon 16. Accordingly, as shown in FIG. 5B, illustrative embodiments may provide a delayed nerve growth factor 60 release profile along the length of the channel 52, such that concentrations nearer to the proximal end 56 (e.g., the first concentration 60A) are released and available to the axon 16 before concentrations near the distal end 58 (e.g., the second concentration 60B). In various embodiments, the release profile along the length of the channel 52 is configured so that a substantial portion (or all) of the growth factor 60 concentration releases in a manner that substantially coincides with axon 16 growth along the longitudinal axis 54.

Alternatively, the nerve growth factor 60 may be released substantially at the same time throughout the entirety of the channel 52 (e.g., within the first 24-hours of implantation). Preferably, when the nerve growth factor 60 is released substantially uniformly (e.g., substantially across the entirety of the length of the channel), the second nerve growth factor concentration 60B has a longer release duration than the first nerve growth factor concentration 60A. As an example, release may start at day 1 and last for approximately 15-30 days (e.g., for the nerve growth factor 60, such as GDNF). However, the released total concentration may be less at the first concentration 60A (e.g., about 0-5 ng/ml) than at the second concentration 60B (e.g., 90-100 ng/ml) at any given day, as it is continuously released and also continuously degraded or up taken by the cells.

As mentioned, various embodiments may modify the drug delivery profile of the nerve growth factor 60 such that the duration of release changes. For example, as shown in FIG. 5C, the available large concentration of nerve growth factor 60 near the distal end 68 may be configured to release over a longer time period than the available concentration of nerve growth factor 60 near the proximal end 56. Accordingly, the release duration may be include a gradient. In some embodiments, the duration of the release for the nerve growth factor 60 may be between about 10 days to about 20 days near the proximal end 56 (e.g., at the first concentration 60A) and may be between about 25 days to about 30 days near the distal end 58 (e.g., at the second concentration 60B).

It should be understood that the some portion of the concentration may be available immediately and some portion of the concentration may be enduring. In various embodiments, this is due to the microparticle degradation rate and therefore the release of the encapsulated factors 60, 62 or the rate of de-sequestration of the factors 60, 62 from the matrix 50.

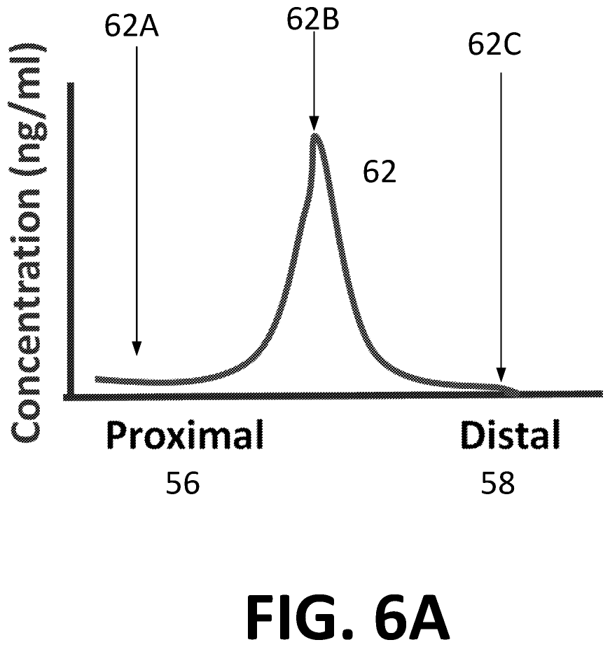

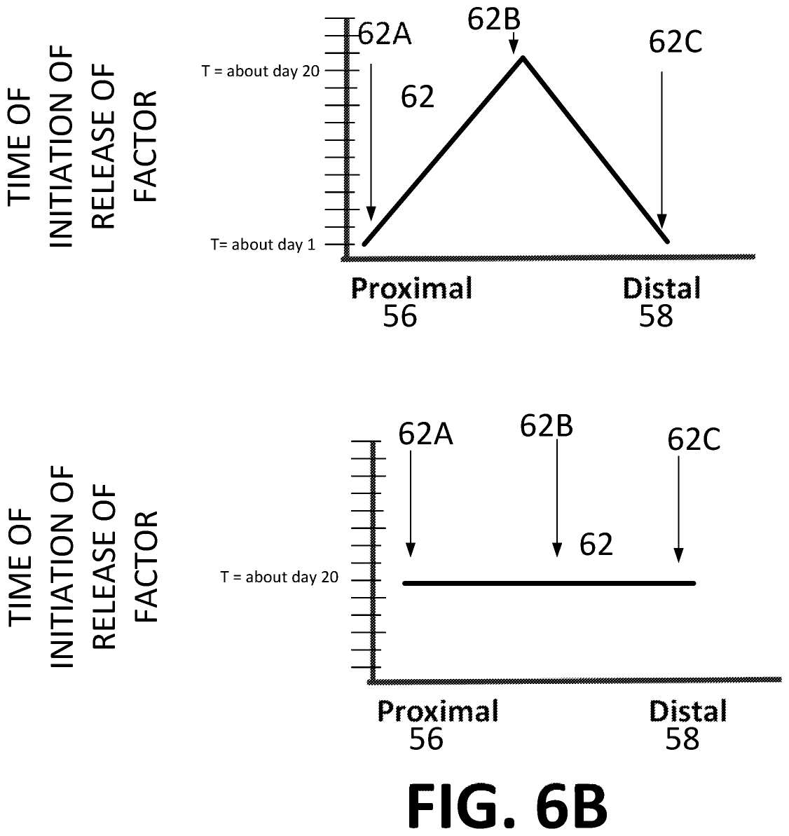

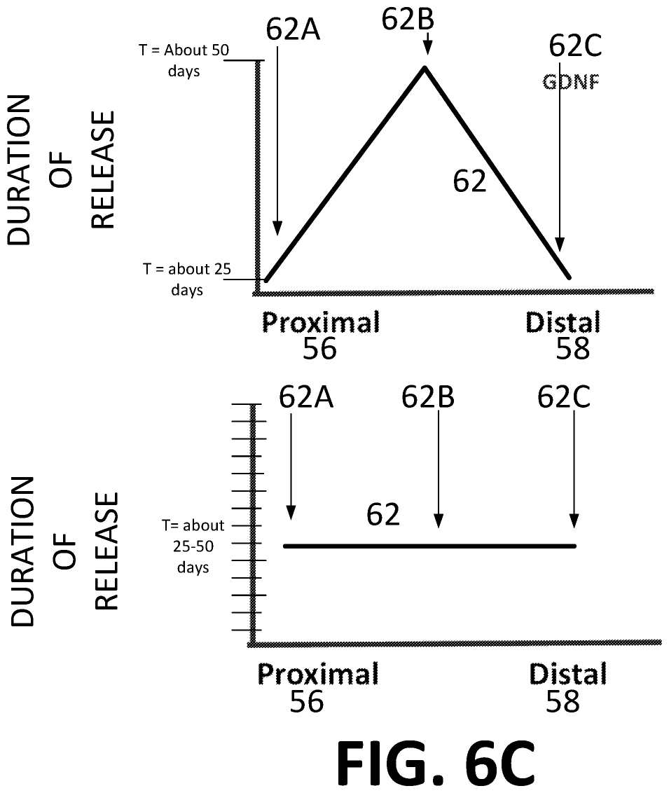

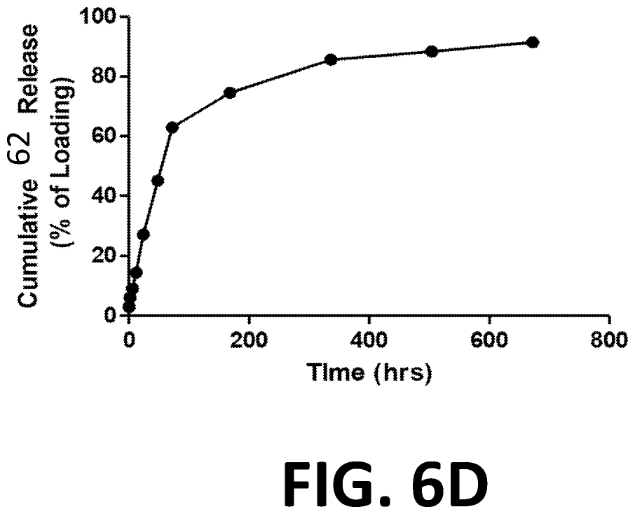

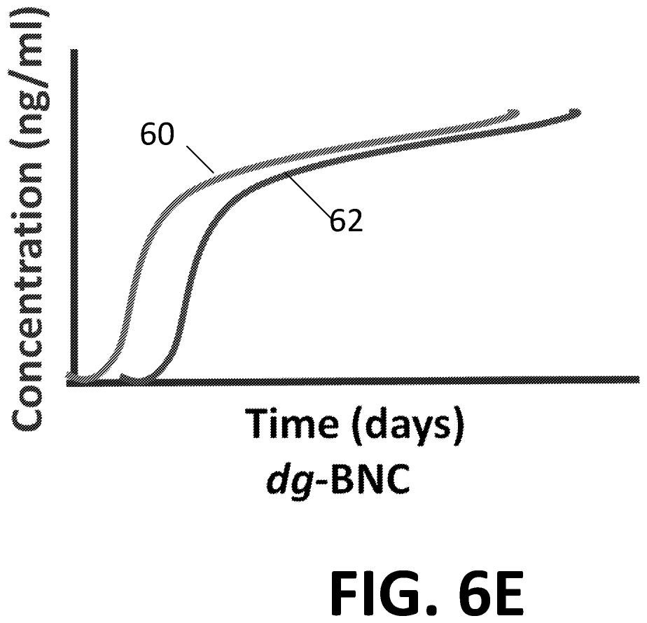

Similar to the nerve growth factor 60, various embodiments have varying concentrations of myelination factor 62 distributed over the length of the channel 52. FIG. 6A graphically shows an example of the concentration of the myelination factor 62 from the proximal end 56 to the distal end 58 in accordance with illustrative embodiments. The myelination factor 62 has a first myelination factor concentration 62A near the proximal end 56, a third myelination factor concentration 62C near the distal end 58, and a second myelination factor concentration 62B between the first concentration 62A and the second concentration 62C. The concentration of myelination factor 62 increases from the first concentration 62A to the second concentration 62B along the longitudinal axis 54, and decreases from the second concentration 62B to the third concentration 62C. Various embodiments therefore provide a concentration gradient that increases towards the middle of the channel 52 and decreases towards the ends of the channel. This type of gradient may be referred to as a centrally peaked gradient. In various embodiments, the concentration of the myelination factor 62 may be about 0-5 ng/ml at the first myelination concentration 62A (e.g., near the proximal end 56), about 15-30 ng/ml at the second concentration (e.g., near the center of the channel 52), and about 0-5 ng/ml at the third concentration 62C (e.g., near the distal end 58). In various embodiments, the myelination factor 62 may include recombinant human NRG1-β1 (Thr176-Lys246, EGF Domain; R&D Systems, MN, USA) and/or the SDMF fragment.

The inventors discovered that a concentration gradient with a peak between two lower concentrations (e.g., near the middle of the length of the length of the channel 52) for the myelination factor 62 advantageously allows for remyelination of the regenerated axons 16 of the nerve 10 across long gaps 28.

From experimentation, the inventors determined that failure to use a myelination factor results in a number of regenerated axons (using the growth factor concentrations discussed above) that are unmyelinated near the distal end. By using myelination factor 62, Schwann cells 66 migrate towards the growing axon 16.

FIG. 6B graphically shows examples of the time of initiation of release of myelination factor in accordance with illustrative embodiments. Various embodiments may provide a continuous release last for approximately 25-50 days for the myelination factor 62.

FIG. 6C graphically shows examples of the duration of release of myelination factor in accordance with illustrative embodiments. In some embodiments, the total duration of release changes from the proximal end to the distal end between the two different spatial gradients, where the total duration of the central gradient is larger than that of the linear gradient. Illustrative is embodiments titer the composition of the co-polymers to adjust the release profile according to the length of the channel, as needed. In various embodiments, the duration of release for the myelination factor 62 is between about 25 days and about 50 days.

FIG. 6D schematically shows an example of a cumulative release pattern for the myelination factor 62 over time in accordance with illustrative embodiments.

FIG. 6E schematically shows an example of a cumulative release pattern for the nerve growth factor 60 and the myelination factor 62 in accordance with illustrative embodiments. As shown, there is a time delay between the release of the growth factor 60 and the myelination factor 62. Despite usage of the time delay, it should be noted that there may be some small unspecified amount of factor 60, 62 (e.g., non-encapsulated) which is available immediately, with additional supply of factor 60, 62 being released over time. This non-substantial release is generally under the concentration of 1 ng/mL, and release of this non-substantial amount of factor 60, 62 is not considered as part of the time delay. Illustrative embodiments release a substantial amount of peptide, protein, drug, or molecule, where substantial release is of a particular concentration (e.g., at least 1-100 ng/mL).

In various embodiments, the nerve growth factor 60 may include nerve growth factor (NGF), Brain-derived neurotrophic factor (BDNF), Glial cell line-derived neurotrophic factor (GDNF) (e.g., for promoting motor neuron growth), Insulin-like growth factor-1 (IGF-1), and Ciliary neurotrophic factor (CNTF), Pleiotrophin (also known as heparin-binding growth factor 8, HBGF-8) (e.g., for promoting sensory neuron growth). Depending on the type of the nerve 10, illustrative embodiments titrate any one of those things and promote mostly sensory or mostly motor.

In various embodiments, the myelination factor 62 may include neuregulin-1 (NRG1, also known as Acetylcholine receptor-inducing activity (ARIA), Breast cancer cell differentiation factor p45, Glial growth factor, Heregulin (HRG), Neu differentiation factor, Sensory and motor neuron-derived factor), platelet-derived growth factor (PDGF), fibroblast growth factor (FGF), and Sonic hedgehog (Shh). Preferably, the myelination factor 62 includes neoregulin1-type3, which is a releasable form that activates the Schwann cells and helps them to become pro-regenerative and pro-myelinating.

While the addition of myelination factor 62 independently assists with myelination of axons 16, the inventors discovered that the longitudinally central peak concentration gradient for myelination factor 62 improves axon 16 myelination relative to a substantially constant longitudinal distribution of myelination factor 62. While a constant myelination factor 62 concentration throughout the length of the channel is operable, most of the grown axons 18 do not become myelinated. The inventors estimate from experimentation that of a possible 15,000-20,000 grown axons, around 100 are myelinated with a constant myelination factor 62 concentration. The inventors suspect, but have not confirmed, that the reason for this unexpected result is due to a process called axon sorting. Myelination factor 62 also assists in axon sorting. When myelination factor 62 is provided, Schwann cells are encouraged to do the sorting—the myelination also requires the axon to be “active” (not growth wise).

From the inventors experiments, it appears that axon grown is time-dependent, in that the myelination factor 62 and the growth factor 60 must be introduced at particular times.

Thus, various embodiments provide a time delay between the release of growth factor and the release of myelination factor 62. For example, the myelination factor 62 may be released 1 week to 4 weeks after growth factor 60 is release begins (e.g., about 18-22 days after growth factor 60 release begins). Preferably, the release of myelination factor is a function of the rate of growth of the axons 16. The axons 16 grow to a point that is near to the middle of the device at different times based on the overall length of the device. For example, the axons 16 get to the middle much faster in a 2 cm long channel than a 6 cm long channel. Illustrative embodiments pro tune the temporal release profile of the factors 60, 62 to account for this temporal difference in axon-arrival time.

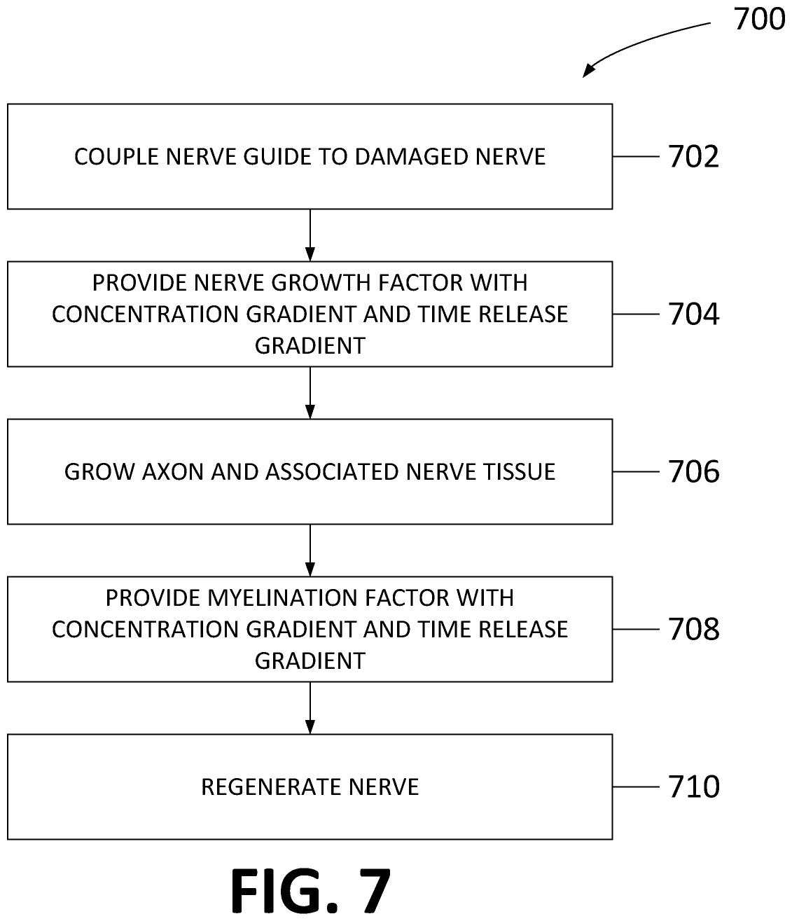

FIG. 7 shows a process of regenerating the nerve 10 in accordance with illustrative embodiments. It should be noted that this method is substantially simplified from a longer process that may normally be used. Accordingly, the method shown in FIG. 7 may have many other steps that those skilled in the art likely would use. In addition, some of the steps may be performed in a different order than that shown, or at the same time. For example, in some embodiments, step 706 may begin in parallel with or before step 702, and may have a longer duration. Furthermore, some of these steps may be optional in some embodiments. Accordingly, the process 700 is merely exemplary of one process in accordance with illustrative embodiments of the invention. Those skilled in the art therefore can modify the process as appropriate.

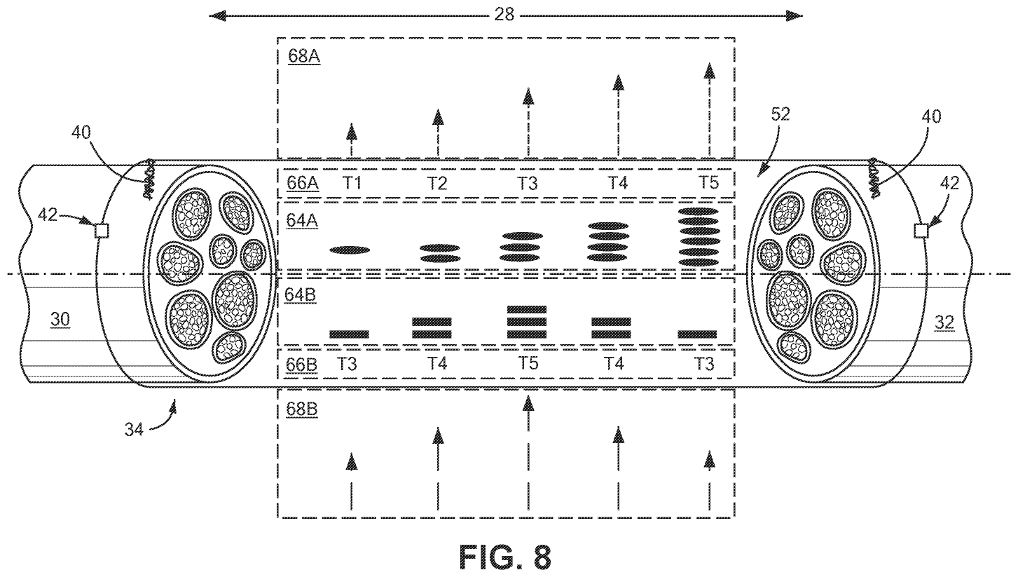

The process of FIG. 7 is described with reference to FIG. 8, which schematically shows the device coupled to the damaged nerve 10 with varying factor concentrations in accordance with illustrative embodiments. FIG. 8 schematically shows an enlarged single channel 52. It should be understood that this is for discussion purposes only, and the device 34 may contain multiple channels 52 having the same or different growth factor 60, 62, growth factor concentrations, and/or release characteristics. Accordingly, for simplicity, the device 34 and the channel 52 are shown as having the same longitudinal axis 48/54, which otherwise would be substantially parallel.

The process begins at step 702, which couples the nerve guide device 34 to the damaged nerve 10. The medical practitioner identifies the length of the gap 28 of the damaged nerve 10, and selects an appropriately sized device 34. To that end, illustrative embodiments may provide a kit with a number of devices 34 that accommodate for varying size gap 28 lengths and/or time elapsed since patient incurred injury (e.g., within 24-hours, within 1-week, etc.). The distribution and release of the factors 60, 62 embedded within the device 34 may be suited to axon 18 growth based on the particular patient situation.

After selection of the appropriate device 34, the surgeon couples the device 34 to the nerve 10. To that end, the device 34 may include an indicator 42 or other marking that indicates the proper orientation of the device 34 relative to the nerve 10 (e.g., proximal side vs. distal side). The surgeon the uses the coupling portion 40 of the device 34 to couple with the respective nerve trunk 30, 32. FIG. 8 shows that the device 34 is sutured to the nerve 10, but any conventional method of attachment to the nerve may be used.

It should be noted that although various embodiments refer to using the device 34, some embodiments may use a similarly configured nerve graft or other scaffolding (e.g., dissolvable) including the factors 60, 62 in the various concentrations and release profiles as described herein.

In various embodiments, the housing 44 is directly coupled to the epineurium 20. Inside of the housing 44 is the matrix 50 defining the one or more channels 52. The channels 52 may be sized similarly to the perineurium 22. However, the channels 52 are not necessarily aligned with the perineurium 22 because of the axons 16 abilities to navigate their way into the channels 52.

It should be understood that various embodiments are not necessarily trying to reconnect the axons 16 on the proximal trunk 30 back to the remaining portions of the axons 16 on the distal trunk 32. The axons 16 of the distal trunk is 32 generally die off because the axon 16 is disconnected from the cell body that provides proteins. However, the other cells in the nerve tissue remain, though some—particularly Schwann cells—can alter their phenotype over time, and some new cells are recruited. Therefore, the device 34 may couple with the various remaining portions of the distal trunk 32, such as the perineurium 22 scaffold. Illustrative embodiments provide a scaffold for the neuron to grow back to the original target. Accordingly, the distal portion of the neuron may be regrown.

After injury, the distal trunk 32 may include all of the nerve tissue except for the axons 16. This is because immune cells remove the distal ends of the axons. Illustrative embodiments use the device to couple the two trunks of the severed nerve. However, the axons of the distal trunk go away, and illustrative embodiments regrow inside the distal trunk.

When the injury happens, the insulating Schwann cells in the distal trunk 32 are reactivated by inflammation, demyelinate, and become pro-regenerative. The Schwann cells have to align, secrete growth factor and promyelination factors. The Schwann cells actively participate in a number of processes, including attracting the axons 16, wrapping around, sorting them out, etc.

In the absence of illustrative embodiments, as time progresses with delayed reconnection/regrowth (e.g., based on gap size—proximal axons grow 1-2 mm/day). By the time the axons 16 reach the distal trunk 32, the portion of the axons 16 still attached to the distal trunk 32 become non-responsive, and don't secrete endogenous growth factors. Illustrative embodiments thus provide the growth factor 60 to help the axons 16 grow. By providing the growth factors 60 in the pathway/scaffold, illustrative embodiments motivate the proximal axon 16 to grow there. Thus, the nerve growth factors 60 help to drive the axons 16, and the myelination factors 62 help the Schwann cells not to quit is prematurely. As discussed below, the factors 60, 62 are provided at different times and with different concentration gradients. Additionally, the Schwann cells that reside further distally in the distal trunk 32 and do not have axon 16 contact for some threshold amount of time become “senescent” and cease producing growth factors and are no longer sufficiently supportive of regenerating axons 16 to enable effective axon 16 regeneration to the target.

At step 704, the nerve growth factor 60 is provided at a given concentration gradient 64A, release time gradient 66A, and/or release rate gradient 68A. As described previously, the concentration gradient 64A is relative to the dimensions of the channel 52 (e.g., the longitudinal axis 54). The initial release time 66A may provide a gradient along the longitudinal axis 48/54 for the nerve factor 60. The release rate gradient 68A defines the rate at which the concentration of factor 60 releases after the initial time of release. Although the release rate 68A is shown as having a gradient, it some embodiments, the release rate 68A may be continuous. In various embodiments, the duration of nerve growth factor 60 release may be approximately 15-30 days after beginning release. As used herein, the term release is considered to encompass substantial release, i.e., of greater than 1 ng/mL.

The nerve growth factor 60 may include, among other things, nerve growth factor (NGF), Brain-derived neurotrophic factor (BDNF), Glial cell line-derived neurotrophic factor (GDNF), Insulin-like growth factor-1 (IGF-1), and Ciliary neurotrophic factor (CNTF), Pleiotrophin (PTN) (also known as heparin-binding growth factor 8, HBGF-8). Various embodiments may include a plurality of nerve growth factors. At least one, and preferably all, of the nerve growth factors each have the proposed spatial and temporal gradients described herein. For example, some embodiments may include GDNF and PTN, at least one of which has concentration gradient and temporal gradient as described herein (preferably both of the nerve growth factors 60 have concentration gradients 64A, time release gradients 66A, and/or release rate gradients 68A as described herein). Various embodiments may use GDNF for motor nerves and PTN for sensory nerves. Some embodiments may include both (e.g., if the nerve 10 has a combination of motor, sensory, and autonomic neurons).

As shown, the growth factor 60 may be provided in increased concentrations along the length of the channel 52. For reasons described previously, the concentration of growth factor 60 may be triggered to release in a time-release manner. For example, the concentrations nearest to the proximal end may be released at an initial time T1, with subsequent release of concentrations occurring at later times along the length of the channel 52 (e.g., represented by times T2, T3, T4, and T5). Furthermore, the release rate 68A of the growth factor 60 may vary along the length of the channel 52. In the example shown, the release rate 68A may be lowest near the proximal end 56, and may escalate towards the distal end 58.

In some embodiments, T1 may correspond to the first day of implantation of the device 34 (i.e., day 1). Thus, in various embodiments, the nerve growth factor 60 may begin to release immediately or within the first 24 hours after implantation.

The process then proceeds to step 706, which grows the axons 16 and associated nerve 10 tissue. In part, this growth is due to the addition of the nerve growth factor 60 from step 704. However, some of the growth may be caused by the body's natural regeneration. When dealing with large gaps 28 (e.g., 2 cm or greater, particularly 4 cm or greater), the body's regenerative capacities may not regenerate axons 16 of sufficient length, even when a simple nerve guide 34 is provided (e.g., without the factors described herein). For particularly long lengths (e.g., 4 cm or greater, particularly 5 cm or greater), illustrative embodiments have both myelination factor 62 and growth factor 60 to properly regenerate fully myelinated axons 16.

The process then proceeds to step 708, which provides the myelination factor 62 at a given concentration gradient and time release gradient. Similar to the growth factor 60, the myelination factor 62 may have its own unique concentration gradient 64B, release time gradient 66B, and/or release rate gradient 68B. The concentration gradient 64B is relative to the dimensions of the channel 52 (e.g., the longitudinal axis 54). The initial release time 66B may provide a gradient along the longitudinal axis 48/54 for the myelination factor 62. The release rate 68B defines the rate at which the concentration of the myelination factor 62 releases after the initial time of release. Although the release rate 68B is shown as having a gradient, it some embodiments, the release rate 68B may be continuous. In various embodiments, the duration of myelination factor 62 release may be approximately 25-50 days after beginning release.

In some embodiments, T3 may correspond to about day 15 to about day after implantation of the device 34. Thus, in various embodiments, the myelination factor 60 may begin to release after axon 16 growth has begun.

The myelination factor 62 may include, among other things, Neuregulin-1 (NRG1 also known as Acetylcholine receptor-inducing activity (ARIA), Breast cancer cell differentiation factor p45, Glial growth factor, Heregulin (HRG), Neu differentiation factor, Sensory and motor neuron-derived factor), Platelet-derived growth factor (PDGF), Fibroblast growth factor (FGF), Sonic hedgehog (Shh). Additionally, the myelination factor 62 may include one or more isoforms that have been classified as type I NRGs (isoforms with an Ig domain and a glycosylation domain, isoforms 1-8), type II NRGs (isoforms with an Ig domain but no glycosylation domain, isoform 9), type III NRGs (isoforms with a Cys-rich domain, isoform 10) and type IV NRGs (isoforms with additional 5′ exons, isoform 11). All these isoforms perform distinct tissue-specific functions. The myelination factor 62 may also include any natural or synthetic ligand for ERBB3 and ERBB4 tyrosine kinase receptors for remyelination and tyrosine kinases that bind to specific neurotrophins with high affinity. For example, there are three known Trk receptors: TrkA, which binds to NGF; TrkB, which binds to BDNF and NT-4/5; and TrkC, which binds to NT-3. Various embodiments may include a plurality of myelination factors 62. At least one, and preferably all, of the myelination factors 62 used each have the proposed spatial and temporal gradients described herein.

As shown, the myelination factor 62 may be provided in varying concentrations along the length of the channel 52. Advantageously, the concentration of myelination factor 62 may be configured to provide a peak concentration 64B near the middle of the channel 52. For myelination factors 62, a centered gradient advantageously causes the Schwann cells from both ends to migrate quickly into the device. This concentration helps the Schwann cells from the proximal trunk 30 and the distal trunk 32 to meet/connect near the center. Along long gaps 28, without the concentration of myelination factor 62 the Schwann cells from the proximal trunk 30 and the distal trunk 32 are not motivated reach and connect, resulting in incompletely or unmyelinated axons 16.

The myelination factor 62 may be triggered to release in a time-release manner. For example, the concentrations nearest to the proximal end may be released at an initial time T3, which is after the initial nerve growth factor 60 release time T1. Subsequent release of concentrations may occur at later times along the length of the channel 52 (e.g., represented by times T3, T4, and T5). Furthermore, the release rate of the myelination factor 62 may vary along the length of the channel 52. In the example shown, the release rate may be lowest near the proximal end 56, and may escalate towards the distal end 58.

In various embodiments, the growth factor 60 and myelination factor 62 are not released at substantially identical times or substantially identical patterns. Illustrative embodiments account for different physiological occurrences at the proximal trunk 30 and the distal trunk 32. In particular, the proximal trunk 30 has contact with the growing axons 16. The myelination factor 62 at the distal end helps recruit Schwann cells to perform remyelination.

In part, the inventors believe the time staggering is beneficial because Schwann cells have multiple modes. It can be disadvantageous to lose myelination factor before there is an axon positioned nearby and ready to be myelinated (e.g., thereby lose your factor supply). Unused factor degrades, diffuses, is uptaken and moved out. If large concentrations of myelination factor are provided well before the axons are sufficiently near to become myelinated, the myelination factor can undesirably change the way the Schwan cells respond. Accordingly, illustrative embodiments delay the release of the myelination factor until the axons are inside the tube and positioned to be myelinated.

Accordingly, the release of the two factors 60, 62 is staggered, and illustrative embodiments provides a sequence of events. First, the axons 16 grow using nerve growth factor 60, and then the grown axons are remyelinated with the assistance of the myelination factor 62. Various embodiments achieve this staggered delivery using a mix of encapsulants/polymer mix controlled delivery, as known by those skilled in the art.

Returning to FIG. 7, the next step 710 regenerates the nerve 10. The regenerated nerve 10 may regenerate to cover gaps 28 of various lengths. Some embodiments may grow and myelinate axons 16 for gaps 28 up to the length from the neural cell body (e.g., near the spinal cord) to the target. Illustrative embodiments enable, in addition to short gap 28 regeneration, long gap 28 regeneration (e.g., 2 cm and greater, 4 cm and greater, or 6 cm and greater). In particular, successful regeneration of the nerve 10 includes regrowth of multiple axons 16 to their original or other suitable target destination, and furthermore myelination of the regrown portion of the axons 16.

Practically, regeneration of the nerve 10 can be tested by evoked sensory function (evoked is distinguished from spontaneous, which could arise from aberrant activity in the injured nerve), motor control (reflexive or volitional), or autonomic function. Tests for determining whether the nerve 10 has regenerated may include, among other things:
Recovery of Static 2-point Discrimination in the Affected Digit;
Changes in PROMIS Pediatric Upper Extremity/Parent Proxy Upper Extremity (PUE/PPUE) outcomes assessment post-operative;
Change in Visual Analogue Scale (VAS) For Pain Score post-operative,
Changes in Reported Pain Using the Faces Pain Scale-Revised Scale (FPS-R) assessment post-operative;
Change in Sensory Function as measured by Medical Research Council Classification (MRCC) for Sensory Recovery;
Change in Motor Function as measured by Medical Research Council Classification (MRCC) for Motor Recovery;
Change in Motor Function as measured by Grip and Pinch; and/or
Change in Tinel's Sign

As an example, assessments for nerve regeneration include Tinel sign, which is an evoked assessment of axonal 16 regeneration within the distal trunk 32. A sensation can be evoked by electrically or mechanically stimulating the nerve segment distal to the repair site. The growing axons 16 are mechano-sensitive, so poking the nerve 10 (e.g., by pushing on the skin) can induce them to fire action potentials. Electrical stimulation can activate the axons 16 wherever they are so one can progress increasingly further distally along the course of the nerve until there is no more sensation evoked. This sign offers an indication of the progress of regeneration. A successful measurable outcome evokes sensation (normal or abnormal) in the body area that was previously innervated by the stimulated nerve (e.g., distal to the cut-end of the proximal stump, including inside the device). There is likely to be sensation evoked from the tissue overlying the regenerating axons in the distal nerve, but those are discounted as not part of the test.

As yet another example, autonomic function (sweating) via galvanic skin response (GSR) may be used to assess nerve regeneration. GSR increases regional temperature to induce a local response in which the autonomic nervous system activates sweat glands in the heated region. This reduces the electrical resistance of the skin, which can be detected (GSR). By heating the denervated region one can measure when, and to what degree, the GSR returns as an indication of regeneration of the injured autonomic axons. A successful measurable outcome includes the presence of sweat in the heated area, as long as the sweating area is one that had been denervated by the injury and is now having restored sweating function.

The process 700 then comes to an end.

It should be understood that in some embodiments, some steps are optional or may be skipped. For example, when dealing with short gap injuries, some embodiment may exclude myelination factor. Sufficiently short gaps 28 is may be able to remyelinate using the bodies naturally regenerative abilities. However, the inventors discovered that nerves 10 may be regenerated across long gaps using a combination of growth factor 60 and myelination factors 62.

Although various embodiments refer to the channel 52 having a given concentration, as discussed previously, the factors 60, 62 may be positioned in a variety of places throughout the device. Thus, discussion with reference to concentrations within the channel 52 are not intended to limit the placement of the factors 60, 62 of various embodiments. In illustrative embodiments, the concentrations of factors 60, 62 described herein may similarly be positioned in any part of the device (e.g., the matrix, the housing, etc.) and/or any part of the device, alone or in combination with another part of the device, may be configured to release the factors 60, 62 into the channel in the prescribed concentrations described herein.

As used in this specification and the claims, the singular forms “a,” “an,” and “the” refer to plural referents unless the context clearly dictates otherwise. For example, reference to “the channel” in the singular includes a plurality of channels, and reference to “the nerve growth factor” in the singular includes one or more nerve growth factors known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A nerve regeneration device comprising:
a nerve guide having a proximal end and a distal end, the nerve guide having a housing with one or more ducts, the one or more ducts extending in a direction that is substantially transverse to a longitudinal axis of the nerve guide, and
a matrix material disposed within the housing, the matrix material defining one or more channels extending in a direction that is substantially along the longitudinal axis of the nerve guide,
wherein the channels comprise channel material containing:
nerve growth factor configured to enhance the growth of axons and associated nerve tissue, wherein the channel has a first growth factor concentration nearer to the proximal end and a second growth factor concentration nearer to the distal end, the second growth factor concentration being higher than the first growth factor concentration;
myelination factor configured to enhance myelination of the grown axons, wherein the channel has a first myelination factor concentration nearer to the proximal end, a third myelination factor concentration nearer to the distal end, and a second myelination factor concentration between the first myelination factor concentration and the third myelination factor concentration, the second myelination factor concentration being higher than the first myelination factor concentration and higher than the third myelination factor concentration.

2. The nerve regeneration device of claim 1, wherein the housing includes a indicator that indicates a proper orientation of the device.

3. The nerve regeneration device of claim 1, wherein the first nerve growth factor concentration is at the proximal end of the channel and the second nerve growth factor concentration is at the distal end of the channel, and
the first myelination factor concentration is at the proximal end of the channel and the second myelination factor concentration is at the middle of the channel, and the third myelination factor is at the distal end of the channel.

4. The nerve regeneration device of claim 1, wherein the channel has a substantially linear gradient of concentration of growth factor along the longitudinal axis of the channel from the first growth factor concentration to the second growth factor concentration.

5. The nerve regeneration device of claim 1, wherein the channel has a substantially central peak gradient of concentration of growth factor along the longitudinal axis of the channel from the first growth factor concentration to the second growth factor concentration.

6. The nerve generation device of claim 1, wherein the device is configured to:
release the nerve growth factor from a biodegradable polymer at a first time for a first duration,
activate the myelination factor at a second time for a second duration,
wherein the second time is after the first time, and the first duration temporally overlaps with the second duration.

7. The nerve regeneration device of claim 6, wherein the second time is at least 2 weeks after the first time.

8. The nerve regeneration device of claim 6, wherein a release rate of the nerve growth factor increases from the first growth factor concentration to the second growth factor concentration.

9. The nerve generation device of claim 1, wherein the channels comprise collagen containing:
a secondary nerve growth factor, wherein the channel has a first secondary growth factor concentration nearer to the proximal end and a second secondary growth factor concentration nearer to the distal end, the second secondary growth factor concentration being higher than the first secondary growth factor concentration;

10. A method of regenerating a damaged nerve having a nerve gap of at least 4 cm, the method comprising:
providing a damaged nerve having a proximal trunk and a distal trunk;
providing a first growth factor concentration nearer to the proximal trunk and a second growth factor concentration nearer to the distal trunk, the second growth factor concentration being higher than the first growth factor concentration;
providing a first myelination factor concentration nearer to the proximal trunk, a third myelination factor concentration nearer to the distal trunk, and a second myelination factor concentration between the first myelination factor concentration and the third myelination factor concentration, the second myelination factor concentration being higher than the first myelination factor concentration and higher than the third myelination factor concentration;
restoring function of the nerve over a gap of at least 4 cm.

11. The method of claim 10, wherein
providing the growth factor begins at a first time,
providing the myelination factor begins at a second time after axons have regenerated to the point at which myelination is possible, and the first time is at least 2 weeks before the second time.

12. The method of claim 10, wherein
providing the nerve growth factor occurs over a first duration,
providing the myelination factor occurs over a second duration, the second duration being longer than the first duration.

13. The method of claim 12, wherein at least 60% of a total concentration of myelination factor is released within 200 hours of coupling the device to the nerve.

14. The method of claim 12, further comprising:
providing a nerve guide having a proximal end and a distal end, the nerve guide having:
a biodegradable housing with one or more ducts, the one or more ducts extending in a direction that is substantially transverse to a longitudinal axis of the nerve guide, and
a matrix material disposed within the housing, the matrix material defining one or more channels extending in a direction that is substantially along the longitudinal axis of the nerve guide.

15. The method of claim 12, further comprising:
providing a first growth factor concentration nearer to the proximal trunk and a second growth factor concentration nearer to the distal trunk, the second growth factor concentration being higher than the first growth factor concentration; and
providing the myelination factor at a third time that is at least 1 week before the second time.

16. The method of claim 14, wherein the matrix comprises agarose.

17. The method of claim 14, wherein the channel comprises collagen.

18. The method of claim 12, wherein growth factor comprises at least one of: nerve growth factor (NGF), brain-derived neurotrophic factor (BDNF), glial cell line-derived neurotrophic factor (GDNF), insulin-like growth factor-1 (IGF-1), and ciliary neurotrophic factor (CNTF), or pleiotrophin (PTN).

19. The method of claim 12, wherein the myelination factor comprises at least one of: Neuregulin-1 (NRG1 also known as acetylcholine receptor-inducing activity (ARIA), breast cancer cell differentiation factor p45, glial growth factor, heregulin (HRG), Neu differentiation factor, sensory and motor neuron-derived factor), platelet-derived growth factor (PDGF), fibroblast growth factor (FGF), or sonic hedgehog (Shh).

20. A nerve regeneration system comprising:
a nerve guide device having a proximal end and a distal end, the nerve guide having a housing with one or more ducts, the one or more ducts extending in a direction that is substantially transverse to a longitudinal axis of the nerve guide, and a matrix material disposed within the housing, the matrix material defining one or more channels extending in a direction that is substantially along the longitudinal axis of the nerve guide;
nerve growth factor configured to enhance the growth of axons and associated nerve tissue,
wherein the device is configured to provide a first growth factor concentration nearer to the proximal end and a second growth factor concentration nearer to the distal end, the second growth factor concentration being higher than the first growth factor concentration;
myelination factor configured to enhance myelination of the grown axons,
wherein the device is configured to provide a first myelination factor concentration nearer to the proximal end, a third myelination factor concentration nearer to the distal end, and a second myelination factor concentration between the first myelination factor concentration and the third myelination factor concentration, the second myelination factor concentration being higher than the first myelination factor concentration and higher than the third myelination factor concentration.
